(19) United States
(12) Patent Application Publication
(10) Pub. No.: US 2023/0056244 A1
(43) Pub. Date: Feb. 23, 2023

(54) MEDICAL DEVICES AND METHODS FOR MAKING MEDICAL DEVICES FOR WARMING A PATIENT

(71) Applicant: GE Precision Healthcare LLC, Wauwatosa, WI (US)

(72) Inventor: Karen P. Becker, Phoenix, MD (US)

(73) Assignee: GE Precision Healthcare LLC, Wauwatosa, WI (US)

(21) Appl. No.: 17/408,011

(22) Filed: Aug. 20, 2021

(51) Int. Cl. A61F 7/08; G16H 10/60; A61G 11/00

(52) U.S. Cl. CPC A61F 7/08; G16H 10/60; A61G 11/00; A61F 2007/0086

(57) ABSTRACT

A medical device for warming a patient. The medical device includes bed configured to support the patient thereon and a heater configured to produce heat for the patient when supported on the bed. A temperature sensor is configured to detect an ambient air temperature apart from the bed. A controller is provided in communication with the temperature sensor, where the controller produces a signal based on the ambient air temperature from the temperature sensor.

### FIELD

The present disclosure generally relates to medical devices for warming patients, and more particularly newborns immediately after birth.

### BACKGROUND

Infants and especially neonates are known to be very sensitive to temperature, and therefore must be carefully monitored and cared for to prevent hypothermia. GE Healthcare® and other medical device manufacturers produce various forms of warmers and incubators for providing warmth to these infants shortly after birth and through the point at which they can effectively regulate their own temperatures. Among these devices are the GE Healthcare's Giraffe and Panda Warmers, the Giraffe Incubator Carestation, Giraffe OmniBed Carestation, and the Giraffe shuttle (which is designed for intrahospital transport with the GE incubators or warmers while in use by a patient). Hybrid devices are also available, such as the OmniBed, which can be converted from a bed to a warmer (open bed) or incubator (closed bed). For simplicity, all medical devices used for warming patients will also be referred to collectively as simply warmers.

According to the World Health Organization (WHO) and International Liaison Committee on Resuscitation (ILCOR) 2020 recommendations, an infant's thermo-regulatory needs require maintaining delivery room temperatures of at least 26° C. Today, between 40% and 85% of infants are admitted to a neonatal intensive care unit (NICU) in a hypothermic state, meaning a body temperature of less than 36.5° C. A hypothermic state has been associated with a 28% risk of mortality and an 11% risk of infection. Particularly in low resourced settings, it has been shown that infants in a hypothermic state for the first 24 hours of life have an 85% mortality rate.

### SUMMARY

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

One example of the present disclosure generally relates to a medical device for warming a patient. The medical device includes bed configured to support the patient thereon and a heater configured to produce heat for the patient when supported on the bed. A temperature sensor is configured to detect an ambient air temperature apart from the bed. A controller is provided in communication with the temperature sensor, where the controller produces a signal based on the ambient air temperature from the temperature sensor.

In certain examples, the heat provided by the heater is controlled based on the signal produced by the controller. In further examples, the heat provided by the heater is also controlled as a function of a gestational age of the patient, a post-conceptual age of the patient, and a birth weight of the patient.

Certain examples further include a display device in communication with the controller, wherein the display device displays the ambient air temperature detected by the temperature sensor. In further examples, the controller is configured to compare the ambient air temperature detected by the temperature sensor to a threshold, and wherein the controller causes the display to produce a warning when the ambient air temperature is below the threshold. In further examples, the display includes a speaker, and wherein the warning is an audible warning. In further examples, the warning includes an indication to increase a temperature of a room in which the temperature sensor is located.

In certain examples, the controller is configured to communicate with an electronic medical record system, and wherein at least one of the ambient air temperature and the comparison of the ambient air temperature to the threshold is provided to the electronic medical record system.

In certain examples, the bed is enclosed with walls and a cover, wherein the heater provides the heat within the walls and the cover, and wherein the ambient air temperature is detected outside the walls and the cover. In further examples, the temperature sensor is a first temperature sensor, further comprising a second temperature sensor that detects a second air temperature that is within the walls and the cover, and wherein the controller also controls the heat provided by the heater based on the second air temperature.

Another example generally relates to a method for making a medical device. The method includes providing a heater configured to produce heat for a patient supported on a bed of the medical device. The method further includes providing a temperature sensor that detects an ambient air temperature apart from the bed. The method further includes providing a controller in communication with the temperature sensor and configuring the controller to produce a signal based on the ambient air temperature from the temperature sensor.

In certain examples, the heat provided by the heater is controlled based on the signal produced by the controller. In further examples, the heat provided by the heater is also controlled as a function of a gestational age of the patient, a post-conceptual age of the patient, and a birth weight of the patient.

Certain examples further include providing a display device and configuring the display device to display the ambient air temperature detected by the temperature sensor. In further examples, the controller is configured to compare the ambient air temperature detected by the temperature sensor to a threshold, and wherein the controller is configured to cause the display to produce a warning when the ambient air temperature is below the threshold. In further examples, the display includes a speaker, and wherein the warning is an audible warning. In further examples, the warning includes an indication to increase a temperature of a room in which the temperature sensor is located.

Certain examples further include configuring the controller to communicate with an electronic medical record system, and further comprising providing at least one of the ambient air temperature and the comparison of the ambient air temperature to the threshold to the electronic medical record system.

Certain examples further include enclosing the bed with walls and a cover, wherein the heater provides the heat within the walls and the cover, and wherein the ambient air temperature is detected outside the walls and the cover.

Another example generally relates to a medical device for an infant patient. The medical device includes bed configured to support a patient thereon and a temperature sensor that detects an ambient air temperature apart from the bed. A heater is configured to produce heat for the infant patient when supported on the bed. A display device displays the ambient air temperature detected by the temperature sensor. A controller is provided in communication with the heater, the temperature sensor, and the display device. The controller is configured to compare the ambient air temperature detected by the temperature sensor to a threshold and to instruct a user to increase the ambient air temperature when below the threshold. The controller controls the heat provided by the heater based on the ambient air temperature from the temperature sensor.

Various other features, objects and advantages of the disclosure will be made apparent from the following description taken together with the drawings.

### BRIEF DESCRIPTION OF THE DRAWINGS

The present disclosure is described with reference to the following drawings.

FIG. 1 is an isometric view of an exemplary infant warmer according to the present disclosure.

FIG. 2 is an isometric view of an exemplary incubator according to the present disclosure.

FIG. 3 is an isometric close up view of a lower portion of a warmer or an incubator such as those shown in FIGS. 1 and 2.

FIG. 4 is a side view of a shuttle connected to the incubator of FIG. 2 for transporting the incubator.

FIG. 5 is a schematic of an exemplary control system configured to produce a signal based on an ambient air temperature according to the present disclosure.

FIG. 6 is a process map for exemplary method for making a medical device according to the present disclosure.

### DETAILED DISCLOSURE

As provided in the Background above, newborn infants are highly susceptible to hypothermia and must be kept at an optimal body temperature. Hypothermia may be especially pronounced for newborn infants of a low body weight or those born before full term, which may require special care from a body heat perspective for an extended period of time (e.g., weeks or months). The present inventor has recognized that the temperature needs of these infants are typically in direct conflict with the needs or desires of healthcare providers or patients in the vicinity. For example, it is common for labor and delivery rooms within a medical facility to be kept at approximately 24° C. This relatively cool temperature is chosen because the laboring mothers and obstetrics staff are consistently too warm during the labor and delivery process. Operating rooms are also typically kept at low temperatures, often as low as 22 degrees C., as this decreases the risk of infection during surgical procedures (along with providing comfort for the staff). Within the context of a mother and her newborn infant, this leads to opposing needs and desires within the same space. Likewise, the inherent differences provide that the medical practitioners on each side (obstetrics staff versus NICU staff) are generally attuned with the needs only of their own direct patients.

In today's practice, the act of adjusting the room temperature from the low temperature desired by the mother or staff to the high temperature needed by the infant relies upon the memory of a staff member. In a best case scenario, one of the obstetrics staff will remember to make this adjustment before the NICU staff arrives, giving the HVAC system time to respond. Likewise, an obstetrics staff member will preferably power on the warmer to ensure a pre-warmed surface before delivery of the infant that the warmer is pre warmed immediately before the birth. However, it is problematic to rely on the memory of the obstetrics staff while they are heavily involved in the intensive and imminent needs of the mother during labor and delivery. Moreover, the obstetrics staff themselves are also most comfortable at the low temperature, encouraging a delay in adjusting the conditions for the infant until absolutely necessary.

Accordingly, the present inventor has recognized that it would be advantageous for the medical device (whether a warmer, an incubator, or other warming device) to assist in reminding the medical practitioners to adjust the temperature of the room, or even to automatically control various operations to ensure warmth for the newborn infant based on the ambient temperature of the room.

Certain infant warmers when enable by the clinician today generate heat for the patient according to a determined “comfort zone”. The comfort zone may include a range of acceptable temperatures for the newborn infant, and/or ranges of acceptable heat provided by a heating device, along with target setpoints thereof. The comfort zone for warmers presently known in the art is determined as a function of the gestational age of the infant, the birth weight of the infant, and the day of life (post-conceptional age) for the infant.

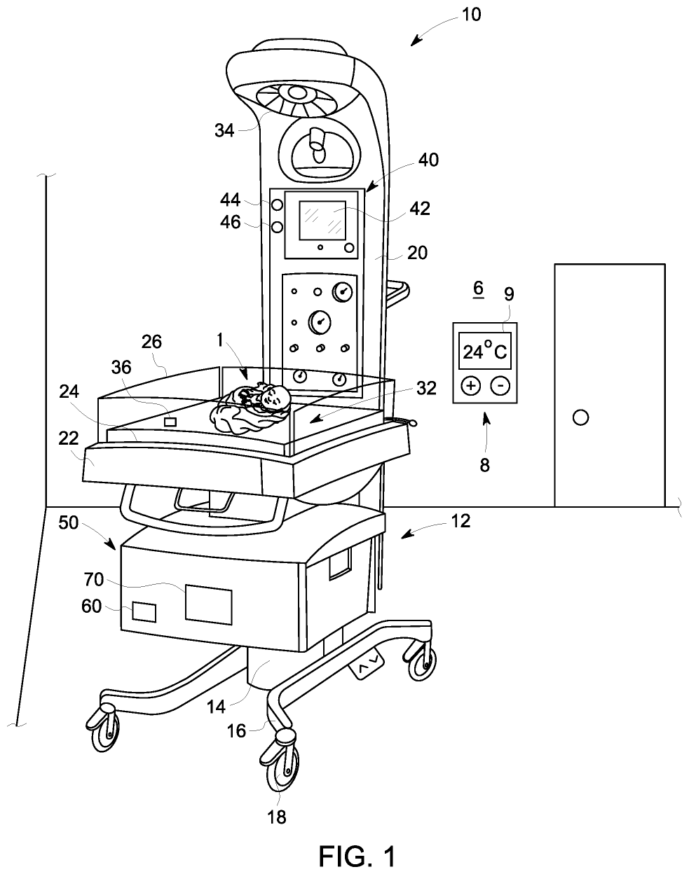

FIG. 1 depicts an exemplary medical device 10 for warming a patient 1 according to the present disclosure. The medical device 10 is shown within a room 2, such as a labor and delivery suite within a medical facility. The ambient air temperature 6 within the room 2 is controlled by room thermostat 8, which is adjustable up and down according to the needs and desires of the patient and medical personnel in a customary manner. The room thermostat 8 also includes a temperature sensor and a display currently showing a temperature reading 9 of 24° C., consistent with the setup of the room 2 during labor and delivery as set by the obstetrics staff.

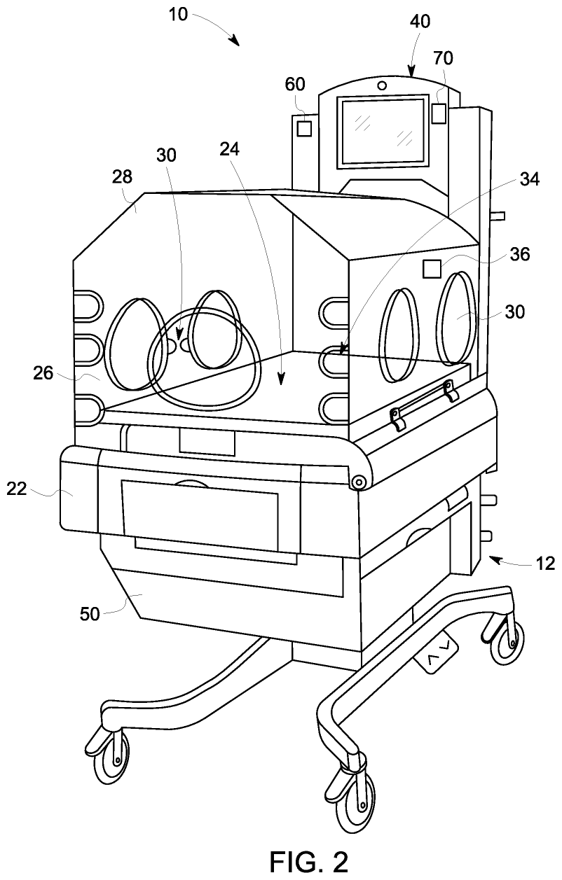

The medical device 10 shown here is an infant warmer having some elements similar to the Giraffe warmer produced by GE Healthcare. The medical device 10 includes a stand 20 supported by legs 14 and feet 16 provided with wheels 18 in a manner presently known in the art. A column 20 extends upwardly from the stand 12 and supports a platform 22 therefrom. A platform 22 may be height adjustable along the column 20 in a manner presently known in the art. The platform 20 is configured to support a bed 24 thereon (here, a mattress), which itself is configured to support the patient 1. Walls 26, and in certain cases a cover 28 (See FIG. 2), generally surround and cover the bed 24, respectively, to prevent the patient 1 from falling from the bed 24 and also to maintain a controlled environment within the interior defined by the walls 26 and cover 28 (in other words, to provide a closed, heated environment).

The air within the interior defined by the walls 26 (and when present, the cover 28) is also referred to as inside air 32. The temperature of the inside air 32 is controlled at least in part by operation of a heater 34. The heater 34 may be a heat generating device such as those used within the exemplary warmers described in the background. For example, the heater 34 may be a radiant heater that generates heat downwardly towards the patient from above, and/or a convective heater may be positioned below the mattress of the bed 24 (e.g., for an incubator), and/or positioned in other locations effective in changing the temperature of the inside air 32. An inside temperature sensor 36 is provided within the interior of the platform 22 (e.g., inside the walls 26) to detect the temperature of the inside air 32 in a manner known in the art. It should be recognized that when no cover 28 is present, the inside air 32 interior is more able to mix with the ambient air 6 within the room 2. For the purpose of this disclosure, the interior air 32 will be assumed to be the volume within the walls 26, and above the walls 26 to include any volume customarily surrounding an infant patient when lying on the bed 24 (e.g., within 2′ or 1′ above the bed 24, for example).

With continued reference to FIG. 1, an enclosure 50 is also supported by the stand 12, which in the present example contains a controller 70 for operating the medical device 10 in a manner presently known in the art. The medical device 10 of FIG. 1 further incorporates an outside temperature sensor 60 at a location other than the interior of the platform 22 defined by the walls 26. In the example shown, this outside temperature sensor 60 is also incorporated within the enclosure 50. The outside temperature sensor 60 detects the ambient air temperature 6 of the room 2 in a similar manner to the temperature sensor within a room thermostat 8 described above. The present disclosure contemplates positioning the outside temperature sensor 60 in differing locations apart from the bed 24, provided it measures the ambient air temperature 6 in the room 2 rather than the temperature of the inside air 24 within the interior of the platform 22 (i.e., that which the patient would experience when lying on the bed 24).

The medical device 10 further includes a user interface 40 for the controlling operations of the medical device 10. The user interface 40 includes a display 42 configured to provide warning indications (colors, icons, and/or the like) as well as messages relating to operation of the medical device 10. A speaker 44 and one or more lights 46 are also provided as part of the user interface 40, which may be separate from the display 42. The speaker 44 and lights 46 may provide further information regarding the operational status of the medical device 10 and/or communicate information to an operator via sounds, spoken text, spoken words, flashing, varying colors, and/or the lights being on or off. In this manner, as is discussed further below, the user interface 40 provides feedback customary of warmers presently known in the art, but also additional information, warnings, and/or the like according to the present disclosure. It should be recognized that the user interface 40 may also or alternatively be provided via an external device (e.g., a mobile device such as a tablet or smart phone) in communication with the medical device. For example, a smart phone may serve as the display 42 (alone or in conjunction with another display 42 on the medical device 10) that communicates with the medical device 10 via Bluetooth® or another wireless protocol known in the art.

FIG. 2 shows a medical device 10 similar to that of FIG. 1, but now as an incubator rather than an infant warmer. The medical device 10 of FIG. 2 includes a cover 28, whereby the interior of the medical device 10 is now defined by the walls 26 and the cover 28. Port holes 30 are defined within the walls 26 and/or cover 28 to provide access to the interior of the platform 22 without having to open one or more of the walls 26 and/or the cover 28 in a manner presently known in the art. In the example shown, the inside temperature sensor 36 is provided on one of the walls 26 with the heater 34 providing a source of heat (e.g., via a heater and blower providing warm air) into the interior through another of the walls 26. In certain examples, the heater 34 is provided below the mattress. However, it should be recognized that the inside temperature sensor 26 may be positioned anywhere in which it can detect the inside air 32 temperature within the interior defined by the walls 26 and the cover 28 (including below or in conjunction with the mattress of the bed 24). In this example, the outside temperature sensor 60 is not provided with the enclosure 50 below the platform 22, but is instead incorporated (as is the controller 70 for operating the medical device 10) with or near the user interface 40.

With reference to FIGS. 1 and 2, the medical device 10 is provided with a heater 34 configured to produce heat for the patient 1 when supported on the bed 24. As discussed above, the heater 34 may be operated at least in part based on the readings of the inside temperature sensor 36 in a manner generally known in the art, and/or based on a set point by the clinical user for the patient. In certain examples, the medical device 10 is configured to operate the heater 34 at a given output based on the calculated comfort zone without the knowledge of, and/or without being based on, the actual temperature of the inside air 32.

Unlike warming devices presently known in the art, the medical devices 10 of FIGS. 1 and 2 operate at least in part based on the readings of the outside temperature sensor 60. In particular, the controller 70 is provided in communication with the outside temperature sensor 60 such that the controller 70 receives the ambient air temperature 6 detected by the outside temperature sensor 60. The controller 70 is further configured to produce a signal based on the ambient air temperature 6 detected by the outside temperature sensor 60.

This signal produced by the controller 70 may be used in one or more different ways to improve the care for the patient 1 with respect to treating and preventing hypothermia. In certain examples according to the present disclosure, the comfort zone or temperature at which the heater 34 heats the interior of the platform 22 for the patient 1 incorporates the ambient air temperature 6 detected by the outside temperature sensor 60. For example, the heater 34 may be controlled by the controller 70 to increase the heat generated for the patient 1 as the ambient air temperature 6 of the room 2 decreases. The present inventor has found that the advantages of this functionality may be even further pronounced for medical devices 10 not having a cover 28, whereby the interior of the platform 22 is more prone to mixing with the cold ambient air temperature 6.

In other examples, the controller 70 is configured to display the ambient air temperature 6 as detected by the outside temperature sensor 60, for example on the display 42, serving as an indicator to medical personnel that the room thermostat 8 may require adjusting. Beyond simply displaying the ambient room temperature 6 on a display 42, additional warnings or messages may appear, which may be triggered by the ambient air temperature 6 being below a predetermined threshold (e.g., 26° C.). The warnings or messages in certain examples include express instructions for medical personnel to increase the temperature within the room 2, which may also alternatively involve sounds or messages from the speaker 44. Other exemplary responses by the controller 70 producing a signal consistent with a low ambient air temperature 6 include activation of lights 46 or changes in audible message, visual color, flash frequency, and/or the like for lights or device display 46.

In certain examples, these warnings or messages also or alternatively appear automatically as soon as the medical device 10 is powered on. This provides early reminders and warnings before the Labor and Delivery or Neonatal staff or infant even arrives such that conditions may be prepared adequately in advance. In further examples, the warnings and/or messages may be automatically cleared when the controller 70 receives a temperature from the outside temperature sensor 60 indicating a sufficiently warm room.

In certain examples, the controller 70 of the medical device 10 also communicates with the room thermostat 8 (via Wi-Fi, Bluetooth®, ZigBee, or other wireless protocols and corresponding hardware as presently known in the art). In these examples, the controller 70 of the medical device 10 can automatically increase the temperature setting of the room thermostat 8 based on a signal that the outside temperature sensor 60 is reading a value below the threshold, for example below 26° C., 25° C., or other pre-set thresholds for operation. In further examples, the controller 70 may read the thermostat 80 in addition to, or in the alternative to using the outside temperature sensor 60 to determine the ambient air temperature 6.

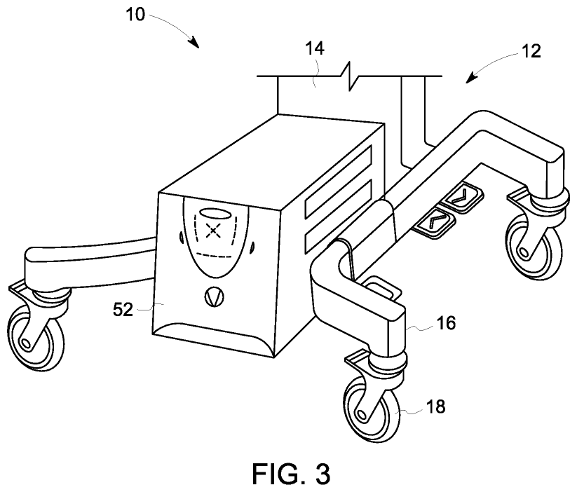

FIG. 3 depicts the lower portion of a medical device 10, and particularly an uninterrupted power supply (UPS) 52 provided therewith in a manner known in the art. In certain examples, outside temperature sensor 60, the controller 70, user interface 40, and/or heater 34 may be wired to operate off of the UPS 52 when power is interrupted. In further examples, the UPS 52 allows the outside temperature sensor 60, the controller 70, user interface 40, and/or heater 34 to operate when the medical device 10 is not plugged into wall power. For example, the user interface 40 may be configured to illuminate a light 46 any time the medical device 10 is in a room that is below the threshold temperature. By operating only limited hardware (e.g., the controller 70, outside temperature sensor 60, and a single light 46), the medical device 10 can operate for long periods of time without separate power, thereby serving as a reminder for staff members to turn up the thermostat 8 of a room 2 any time the medical device 10 is placed in a room 2 in which the temperature is insufficient for its use with a patient.

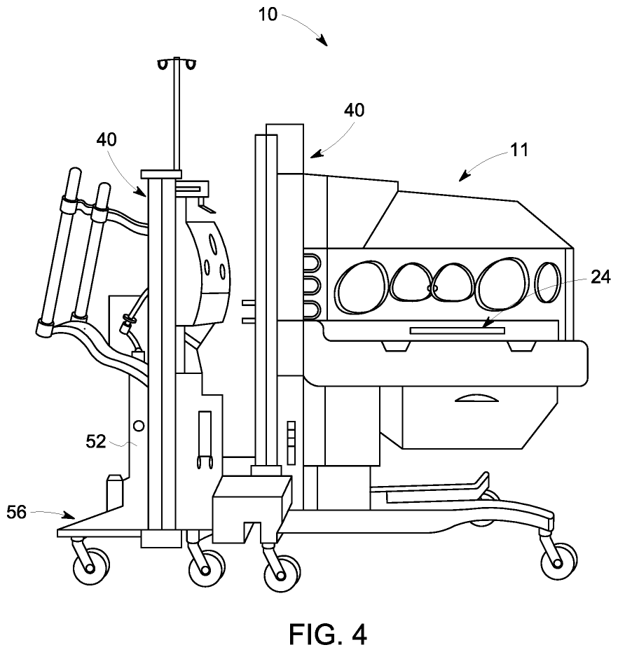

FIG. 4 shows a shuttle device 56 that is configured to connect to the medical device 10 for transporting thereof in a manner known in the art. The shuttle device 56 also includes a UPS 52 or other battery supply as discussed above, which in certain examples provides extended battery life to the medical device 10 itself. This provides extended operation of the features disclosed herein, whether providing addition heat for the patient and/or warnings or other responses to low ambient air temperature 6 conditions.

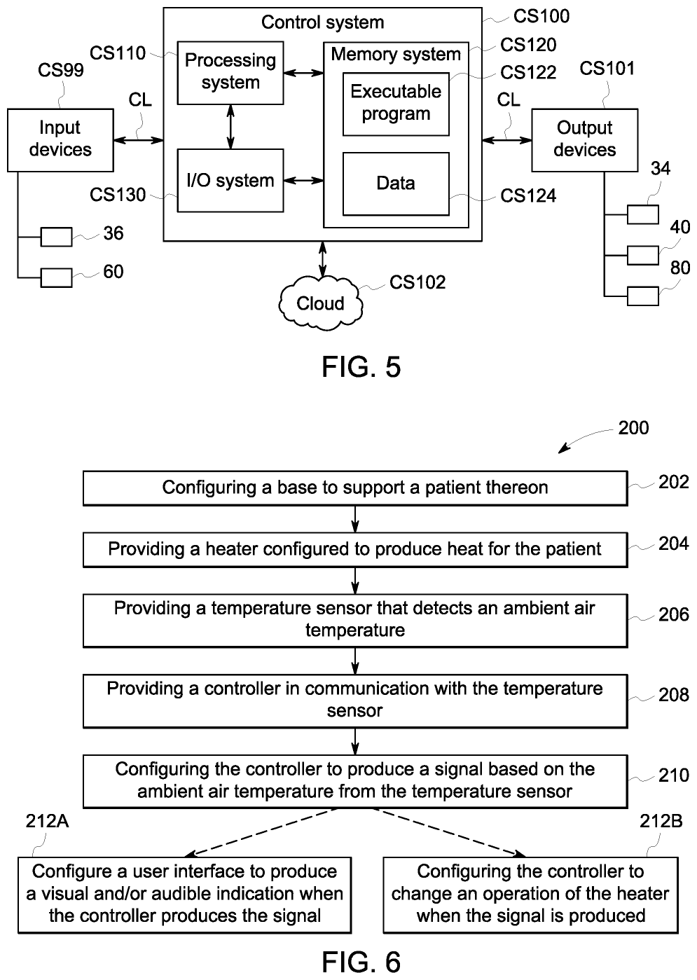

FIG. 5 depicts an exemplary control system CS100 according to the present disclosure, which may be used as the controller 70, for example. Certain aspects of the present disclosure are described or depicted as functional and/or logical block components or processing steps, which may be performed by any number of hardware, software, and/or firmware components configured to perform the specified functions. For example, certain embodiments employ integrated circuit components, such as memory elements, digital signal processing elements, logic elements, look-up tables, or the like, configured to carry out a variety of functions under the control of one or more processors or other control devices. The connections between functional and logical block components are merely exemplary, which may be direct or indirect, and may follow alternate pathways.

In certain examples, the control system CS100 communicates with each of the one or more components of the medical device 10 via a communication link CL, which can be any wired or wireless link. The control module CS100 is capable of receiving information and/or controlling one or more operational characteristics of the medical device 10 and its various sub-systems by sending and receiving control signals via the communication links CL. In one example, the communication link CL is a controller area network (CAN) bus; however, other types of links could be used. It will be recognized that the extent of connections and the communication links CL may in fact be one or more shared connections, or links, among some or all of the components in the medical device 10. Moreover, the communication link CL lines are meant only to demonstrate that the various control elements are capable of communicating with one another, and do not represent actual wiring connections between the various elements, nor do they represent the only paths of communication between the elements. Additionally, the medical device 10 may incorporate various types of communication devices and systems, and thus the illustrated communication links CL may in fact represent various different types of wireless and/or wired data communication systems.

The control system CS100 may be a computing system that includes a processing system CS110, memory system CS120, and input/output (I/O) system CS130 for communicating with other devices, such as input devices CS99 (e.g., the inside temperature sensor 36, outside temperature sensor 60, and/or thermostat 8) and output devices CS101 (e.g., the heater 34, user interface 40, EMR 80 discussed below, and/or thermostat 8), either of which may also or alternatively be stored in a cloud 1002. The processing system CS110 loads and executes an executable program CS122 from the memory system CS120, accesses data CS124 stored within the memory system CS120, and directs the system 10 to operate as described in further detail below.

The processing system CS110 may be implemented as a single microprocessor or other circuitry, or be distributed across multiple processing devices or sub-systems that cooperate to execute the executable program CS122 from the memory system CS120. Non-limiting examples of the processing system include general purpose central processing units, application specific processors, and logic devices.

The memory system CS120 may comprise any storage media readable by the processing system CS110 and capable of storing the executable program CS122 and/or data CS124. The memory system CS120 may be implemented as a single storage device, or be distributed across multiple storage devices or sub-systems that cooperate to store computer readable instructions, data structures, program modules, or other data. The memory system CS120 may include volatile and/or non-volatile systems, and may include removable and/or non-removable media implemented in any method or technology for storage of information. The storage media may include non-transitory and/or transitory storage media, including random access memory, read only memory, magnetic discs, optical discs, flash memory, virtual memory, and non-virtual memory, magnetic storage devices, or any other medium which can be used to store information and be accessed by an instruction execution system, for example.

In certain examples (e.g., powering on the device in a Labor and Delivery setting in preparation for a birth), the medical device 10 is further configured to communicate with an electronic medical record using protocols presently known in the art, including the HL7 communication standard. In particular, the medical device 10 may be configured to communicate the temperature readings from the inside temperature sensor 36 and/or the outside temperature sensor 60 to the EMR 80 to provide a permanent record for the patient 1. This information may be helpful for a clinician later reviewing the case of a patient 1, for example seeing that the outside temperature sensor 60 of the medical device 10 is reading a low temperature such as 24 degrees Celsius at the time of birth , for example.

In certain examples in which the medical device 10 communicates directly with the room thermostat 8, an outside temperature sensor 60 may not be necessary, as the ambient air temperature 6 may be directly read by the medical device 10 via the room thermostat 8. However, the present inventor has recognized that in certain examples it is still beneficial for the outside temperature sensor 60 to be provided with the medical device 10. For example, the outside temperature sensor 60 may control operation of the medical device 10 (including generating warnings, messages, and/or the like) while the medical device 10 is in transit, for example to move the patient from a labor or delivery room to the NIC or elsewhere. In this case, operation of the medical device 10 may be provided by the UPS 52 (see FIG. 3) or shuttle device 56 (see FIG. 4) while the medical device 10 is unable to be plugged into wall power, for example.

FIG. 6 depicts a process flow for an exemplary method 200 for making a medical device according to the present disclosure, including those previously described above. Step 202 provides for configuring a base to support a patient thereon, whereby the base may include a stand 12, legs 14, feet 16, wheels 18, and/or column 20 as described above. Step 204 provides for providing a heater configured to produce heat for the patient, as well as in step 206 providing a temperature sensor that detects an ambient air temperature. A controller is provided at step 208 in communication with the temperature sensor, whereby the controller is configured in step 210 to produce a signal based on the ambient air temperature detected by the temperature sensor. In certain examples, the signal is produced only after the controller compares the ambient air temperature to a predetermined threshold (e.g., 26° C. or 28° C.) and determines that the ambient air temperature is insufficiently low.

In the example of FIG. 6, one or both of two exemplary outcomes may result from the signal produced by the controller in step 210. In the first example of step 212a, a user interface is configured to produce a visual and/or audible indication when the controller produces the signal. In addition or in the alternative, step 212b provides for configuring the controller to change in operation of the heater when the signal is produced by the controller. In certain examples, the heater at step 212b refers to the heater 34 of the medical device 10 itself. However, it should be recognized that the heater may also alternatively refer to the heat generated within the room 2, including that controlled by the room thermostat 8 (see FIG. 1).

The functional block diagrams, operational sequences, and flow diagrams provided in the Figures are representative of exemplary architectures, environments, and methodologies for performing novel aspects of the disclosure. While, for purposes of simplicity of explanation, the methodologies included herein may be in the form of a functional diagram, operational sequence, or flow diagram, and may be described as a series of acts, it is to be understood and appreciated that the methodologies are not limited by the order of acts, as some acts may, in accordance therewith, occur in a different order and/or concurrently with other acts from that shown and described herein. For example, those skilled in the art will understand and appreciate that a methodology can alternatively be represented as a series of interrelated states or events, such as in a state diagram. Moreover, not all acts illustrated in a methodology may be required for a novel implementation.

This written description uses examples to disclose the invention, including the best mode, and also to enable any person skilled in the art to make and use the invention. Certain terms have been used for brevity, clarity, and understanding. No unnecessary limitations are to be inferred therefrom beyond the requirement of the prior art because such terms are used for descriptive purposes only and are intended to be broadly construed. The patentable scope of the invention is defined by the claims and may include other examples that occur to those skilled in the art. Such other examples are intended to be within the scope of the claims if they have features or structural elements that do not differ from the literal language of the claims, or if they include equivalent features or structural elements with insubstantial differences from the literal languages of the claims.

What is claimed is:

1. A medical device for warming a patient, the medical device comprising:
a bed configured to support the patient thereon;
a heater configured to produce heat for the patient when supported on the bed;
a temperature sensor that detects an ambient air temperature apart from the bed; and
a controller in communication with the temperature sensor, wherein the controller produces a signal based on the ambient air temperature from the temperature sensor.

2. The medical device according to claim 1, wherein the heat provided by the heater is controlled based on the signal produced by the controller.

3. The medical device according to claim 2, wherein the heat provided by the heater is also controlled as a function of a gestational age of the patient, a post-conceptual age of the patient, and a birth weight of the patient.

4. The medical device according to claim 1, further comprising a display device in communication with the controller, wherein the display device displays the ambient air temperature detected by the temperature sensor.

5. The medical device according to claim 4, wherein the controller is configured to compare the ambient air temperature detected by the temperature sensor to a threshold, and wherein the controller causes the display to produce a warning when the ambient air temperature is below the threshold.

6. The medical device according to claim 5, wherein the display includes a speaker, and wherein the warning is an audible warning.

7. The medical device according to claim 5, wherein the warning includes an indication to increase a temperature of a room in which the temperature sensor is located.

8. The medical device according to claim 1, wherein the controller is configured to communicate with an electronic medical record system, and wherein at least one of the ambient air temperature and the comparison of the ambient air temperature to the threshold is provided to the electronic medical record system.

9. The medical device according to claim 1, wherein the bed is enclosed with walls and a cover, wherein the heater provides the heat within the walls and the cover, and wherein the ambient air temperature is detected outside the walls and the cover.

10. The medical device according to claim 9, wherein the temperature sensor is a first temperature sensor, further comprising a second temperature sensor that detects a second air temperature that is within the walls and the cover, and wherein the controller also controls the heat provided by the heater based on the second air temperature.

11. A method for making a medical device, the method comprising:
providing a heater configured to produce heat for a patient supported on a bed of the medical device;
providing a temperature sensor that detects an ambient air temperature apart from the bed;
providing a controller in communication with the temperature sensor; and
configuring the controller to produce a signal based on the ambient air temperature from the temperature sensor.

12. The method according to claim 11, wherein the heat provided by the heater is controlled based on the signal produced by the controller.

13. The method according to claim 12, wherein the heat provided by the heater is also controlled as a function of a gestational age of the patient, a post-conceptual age of the patient, and a birth weight of the patient.

14. The method according to claim 11, further comprising providing a display device and configuring the display device to display the ambient air temperature detected by the temperature sensor.

15. The method according to claim 14, wherein the controller is configured to compare the ambient air temperature detected by the temperature sensor to a threshold, and wherein the controller is configured to cause the display to produce a warning when the ambient air temperature is below the threshold.

16. The method according to claim 15, wherein the display includes a speaker, and wherein the warning is an audible warning.

17. The method according to claim 15, wherein the warning includes an indication to increase a temperature of a room in which the temperature sensor is located.

18. The method according to claim 11, further comprising configuring the controller to communicate with an electronic medical record system, and further comprising providing at least one of the ambient air temperature and the comparison of the ambient air temperature to the threshold to the electronic medical record system.

19. The method according to claim 11, further comprising enclosing the bed with walls and a cover, wherein the heater provides the heat within the walls and the cover, and wherein the ambient air temperature is detected outside the walls and the cover.

20. A medical device for an infant patient, the medical device comprising:
a bed configured to support a patient thereon;
a temperature sensor that detects an ambient air temperature apart from the bed;
a heater configured to produce heat for the infant patient when supported on the bed;
a display device that displays the ambient air temperature detected by the temperature sensor; and
a controller in communication with the heater, the temperature sensor, and the display device, wherein the controller is configured to compare the ambient air temperature detected by the temperature sensor to a threshold and to instruct a user to increase the ambient air temperature when below the threshold, and wherein the controller controls the heat provided by the heater based on the ambient air temperature from the temperature sensor.
